(19) United States
(12) Patent Application Publication
(10) Pub. No.: US 2013/0144181 A1
(43) Pub. Date: Jun. 6, 2013

(54) MEASUREMENTS OF FATIGUE LEVEL USING HEART RATE VARIABILITY DATA

(71) Applicants: Donovan L. Fogt, San Antonio, TX (US); John E. Kalns, San Antonio, TX (US); Darren J. Michael, San Antonio, TX (US); William H. Cooke, San Antonio, TX (US)

(72) Inventors: Donovan L. Fogt, San Antonio, TX (US); John E. Kalns, San Antonio, TX (US); Darren J. Michael, San Antonio, TX (US); William H. Cooke, San Antonio, TX (US)

(21) Appl. No.: 13/640,710

(22) PCT Filed: Apr. 14, 2011
(86) PCT No.: PCT/US11/32536

§ 371 Date: Feb. 20, 2013

Related U.S. Application Data
(60) Provisional application No. 61/324,106, filed on Apr. 14, 2010.

(51) Int. Cl. A61B 5/024; A61B 5/0245; A61B 5/00; A61B 5/0456; A61B 5/04

(52) U.S. Cl. CPC A61B 5/02405; A61B 5/0456; A61B 5/04018; A61B 5/746; A61B 5/6831; A61B 5/0006; A61B 5/02455
USPC 600/521; 600/509

(57) ABSTRACT

Methods, apparatuses, and systems for quantifying fatigue of a subject are disclosed. The methods may include measuring an electrocardiogram (ECG) signal from the subject. The methods may further include calculating, with a processing device, a Heart Rate Variability (HRV) metric in response to the ECG signal. The methods may additionally include calculating, with a processing device, a fatigue level in response to the HRV metrics.

The present application claims benefit of priority to U.S. Provisional Application Ser. No. 61/324,106 filed Apr. 14, 2010, the entire contents of which are hereby incorporated by reference.

This invention was made with Government support under contract W91B9462431497 awarded to Hyperion Biotechnology, Inc. by the U.S. Army. The Government has certain rights in the invention.

### BACKGROUND OF THE INVENTION

1. Field of the Invention

The present invention relates generally to the fields of understanding the human nervous system. More particularly, it concerns the measurement of fatigue level using heart rate variability data.

2. Description of the Related Art

Physiologic reserve deteriorates with overwhelming stress, resulting in fatigue characterized by mental or physical exhaustion. Fatigue arising from sleep deprivation profoundly affects cognitive executive functions, and is particularly detrimental to tasks that depend strongly on attention, i.e., tasks requiring an individual to remain focused and on-task rather than rote tasks requiring well-learned, automatic responses (Heslegrave, 1985). While a cognitive test can indicate when the level of cumulative fatigue affects performance, there is currently no continuous, real-time objective indicator of fatigue level preceding a drop in cognitive performance. Sleep-deprived thalamic activation seems to inversely correlate with prefrontal activation (Tomasi et al., 2009) suggesting that under sleep-deprived conditions, more thalamic resources might be required to maintain attention with increasing levels of fatigue. This, in turn, may lead to an impairment of essential networks required for accurate visuospatial attention performance (Chuah et al., 2008; Tomasi et al., 2009). Therefore, it is not surprising that sleep deprivation-related visual performance decrements are followed by decreases in eye-hand coordination. Evaluation of military-related sleep deprivation fatigue has shown that decreased vigilance, mood changes, perceptual and cognitive decrements (Krueger, 1990 (finding sustained military performance in continuous operations: combatant fatigue, rest and sleep needs)) are closely followed by compromised marksmanship (McLellan et al., 2005 (finding caffeine maintains vigilance and improves run time during night operations for Special Forces); Tharion et al., 2003 (finding caffeine effects on marksmanship during high-stress military training with 72 hour sleep deprivation)).

The autonomic nervous system normally compensates for fatiguing stressors by modulating the balance between parasympathetic and sympathetic nervous system cardiovascular control mechanisms. Understanding such autonomic compensatory balance can provide insight into early and sensitive changes in physiological status. For instance, a robust, sympathetically-mediated response to stress is appropriate and beneficial with respect to accommodation of the challenge. However, a parasympathetic predominance under stress reflects an inappropriate response, indicating a progression towards a state of decompensation and failure of physiological function.

High HRV at rest is normal in young, healthy individuals, while reduced HRV has been associated with aging, disease, injury, and increased mortality (Cooke et al., 2006b; Hon and Lee, 1963; Korach, 2001; Riodan et al., 2009). To date, no study has assessed resting HRV throughout a protocol designed to gradually increase fatigue arising from different stressors. Furthermore, no effort has been made to define the relationship between HRV and the progression of fatigue.

Because the causes of fatigue are numerous, asymmetrical, and unpredictable—especially in demanding military or civilian environments, there is a clear need to evaluate the effects of sleep deprivation in combination with measured exposure to additional common stressors, such as physical activity and dehydration. Certain disclosed embodiments demonstrate that fatigue arising from different concurrent stressors is readily identifiable with standard measures of HRV.

### SUMMARY OF THE INVENTION

The present invention provides for methods, apparatuses, and systems for quantifying level of fatigue in a subject that are based on measures of heart rate variability (HRV). The present inventors have found that certain measures of HRV correlate with level of fatigue in subjects.

A method for quantifying fatigue of a subject is disclosed. The method may include measuring an electrocardiogram (ECG) signal from the subject. The method may further include calculating a Heart Rate Variability (HRV) metric derived from the ECG signal. The method may additionally include calculating a fatigue level in response to the HRV metric. A processing device may be used for any of these calculations.

In certain embodiments, the method further includes transmitting the ECG signal to a processing device after measuring the ECG signal from the subject.

In certain embodiments, the method further includes triggering an alarm in response to a particular fatigue level. In certain embodiments, the method further includes subjecting the subject to a stressor and assessing change in the HRV metric versus decline in cognitive performance.

In certain embodiments, calculating the HRV metric includes determining the average R-R interval over a period of time. This period of time may be 30 seconds to 15 minutes. In certain embodiments, calculating the HRV metric includes determining the R-R interval standard deviation over a period of time. This period of time may be 30 seconds to 15 minutes.

In certain embodiments, calculating the HRV metric includes calculating the power spectral density of the ECG signal. Calculating the power spectral density may include filtering the ECG signal with a low-pass impulse response filter to form a filtered ECG signal; and performing a Fourier transform on the filtered ECG signal to form a processed ECG signal. The low-pass impulse response filter may have a cut-off frequency of 0.5 Hz. In certain embodiments, the Fourier transform may have a Hanning window. Calculating the HRV metric may also include calculating the power spectral density of the ECG signal across a frequency range from 0.04 Hz to 0.15 Hz. Calculating the HRV metric may also include calculating the power spectral density of the ECG signal across a frequency range from 0.15 Hz to 0.4 Hz. In certain embodiments, measuring the ECG signal may include an analog to digital conversion.

An apparatus for quantifying fatigue of a subject is also disclosed. The apparatus may include two or more ECG measuring pads configured to measure an ECG signal from a subject. The apparatus may further include a processing device. The processing device may be configured to calculate a Heart Rate Variability (HRV) metric in response to the ECG signal and to calculate a fatigue level in response to the HRV metric. The ECG measuring pad and the processing device may be included within a strap or pad, and the ECG measuring pad may be configured to be positioned in contact with a surface of the subject.

In certain embodiments, the apparatus may further include a transmitting device configured to send the ECG signal to the processing device. The apparatus may also include an alarm configured to trigger a response to the fatigue level. In certain embodiments, configuring the alarm further includes subjecting the subject to a stressor and assessing change in the HRV metric versus decline in cognitive performance, thereby establishing the magnitude of change in HRV metric associated with a particular level of decline in cognitive performance.

In a preferred embodiment, the apparatus may include two ECG measuring pads. The two or more ECG pads of the apparatus may be included in a chest strap.

In certain embodiments of the apparatus, calculating the HRV metric may include determining the average R-R interval over a period of time. The period of time may be 30 seconds to 15 minutes. In certain embodiments of the apparatus, calculating the HRV metric may include determining the R-R interval standard deviation over a period of time. The period of time may be 30 seconds to 15 minutes. The calculation may optionally be repeated.

In certain embodiments of the apparatus, calculating the HRV metric may include determining the power spectral density of the ECG signal. Calculating the power spectral density may include filtering the ECG signal with a low-pass impulse response filter to form a filtered ECG signal and performing a Fourier transform on the filtered ECG signal to form a processed ECG signal. The low-pass impulse response filter may have a cut-off frequency of 0.5 Hz. The Fourier transform may include a Hanning window.

In certain embodiments, calculating the HRV metric includes calculating the power spectral density of the ECG signal across a frequency range from 0.04 Hz to 0.15 Hz. In certain embodiments, calculating the HRV metric comprises calculating the power spectral density of the ECG signal across a frequency range from 0.15 Hz to 0.4 Hz. In certain embodiments, the apparatus comprises an analog-to-digital converter.

A system for quantifying fatigue of a subject is also disclosed. In certain embodiments, the system includes two or more ECG measuring pads configured to measure an ECG signal from a subject. The system may further include a processing device. The processing device may be configured to calculate a Heart Rate Variability (HRV) metric in response to the ECG signal and calculate a fatigue level in response to the HRV metric.

In certain embodiments, the system may also include a transmitting device configured to send the ECG signal to the processing device. In certain embodiments of the system, the two or more ECG measuring pads and the transmitting device are comprised in a first strap or pad. The ECG measuring pad may be configured to be positioned in contact with a surface of the subject.

In certain embodiments, the processing device is comprised in a second strap or pad. In other embodiments, the processing device is comprised in a personal computing device.

In certain embodiments, the system may include an alarm configured to trigger a response to the fatigue level. In certain embodiments, the two or more ECG measuring pads may be comprised in a chest strap. In certain embodiments, the implementation of the system further includes subjecting the subject to a stressor and assessing change in the HRV metric versus decline in cognitive performance, thereby establishing the magnitude of change in HRV metric associated with a particular level of decline in cognitive performance.

In certain embodiments of the system, calculating the HRV metric may include determining the average R-R interval over a period of time. The period of time may be 30 seconds to 15 minutes. In certain embodiments of the system, calculating the HRV metric comprises determining the R-R interval standard deviation over a period of time. The period of time may be 30 seconds to 15 minutes.

In certain embodiments of the system, calculating the HRV metric may include determining the power spectral density of the ECG signal. Calculating the power spectral density may include filtering the ECG signal with a low-pass impulse response filter to form a filtered ECG signal and performing a Fourier transform on the filtered ECG signal to form a processed ECG signal. In certain embodiments, the low-pass impulse response filter may have a cut-off frequency of 0.5 Hz. In certain embodiments, the Fourier transform may further include a Hanning window. In certain embodiments of the system, calculating the HRV metric may include calculating the power spectral density of the ECG signal across a frequency range from 0.04 Hz to 0.15 Hz. In certain embodiments of the system, calculating the HRV metric comprises calculating the power spectral density of the ECG signal across a frequency range from 0.15 Hz to 0.4 Hz. Certain embodiments of the system may further include an analog-to-digital converter.

It is specifically contemplated that any limitation discussed with respect to one embodiment of the invention may apply to any other embodiment of the invention. Furthermore, any composition of the invention may be used in any method of the invention, and any method of the invention may be used to produce or to utilize any composition of the invention.

The use of the term “or” in the claims is used to mean “and/or” unless explicitly indicated to refer to alternatives only or the alternatives are mutually exclusive, although the disclosure supports a definition that refers to only alternatives and to “and/or.”

Throughout this application, the term “about” is used to indicate that a value includes the standard deviation of error for the device and/or method being employed to determine the value.

As used herein the specification, “a” or “an” may mean one or more, unless clearly indicated otherwise. As used herein in the claim(s), when used in conjunction with the word “comprising,” the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE FIGURES

The following figures form part of the present specification and are included to further demonstrate certain aspects of the present invention. The invention may be better understood by reference to one or more of these drawings in combination with the detailed description of specific embodiments presented herein.

FIG. 1. illustrates summary of the experiment performed in Example 1.

FIG. 2 includes graphs illustrating the widely varying levels of fatigue of subjects in Example 1.

FIG. 3 includes graphs illustrating the cognitive performance decrease of the subjects in Example 1.

FIG. 4 includes graphs illustrating the linear mixed-effects modeling of the correlation between fatigue and cognitive performance for the subjects in Example 1.

FIG. 5 includes graphs illustrating the correlation between RRISD and fatigue level for the subjects in Example 1.

FIG. 6 includes graphs illustrating the linear-mixed effects modeling of the correlation between fatigue and RRILF for the subjects in Example 1.

FIG. 7 illustrates embodiments of a method for quantifying fatigue of a subject.

FIG. 8 illustrates an embodiment of an apparatus for quantifying fatigue of a subject.

FIG. 9 illustrates an embodiment of a system for quantifying fatigue of a subject.

### DESCRIPTION OF ILLUSTRATIVE EMBODIMENTS

The present invention is based on the finding that fatigue arising from different concurrent stressors is readily measurable with standard measures of HRV. The findings indicate that simple HRV metrics can be used to predict the progressive increase in subjective fatigue level which, in turn, corresponds to a concomitant decrease in cognitive performance. Early detection of fatigue “risk” in stressed subjects, such as in personnel in demanding military or civilian environments, would allow for preventive strategies to circumvent cognitive or physical performance decrements.

“Fatigue” is defined herein as a state in which the body can no longer consistently maintain a desired or appropriate level of performance on a defined task. The said task can still be performed, albeit at a substandard level. The definition of “fatigue” is not to be confused with “failure”, in which the task can no longer be performed at all. When the body's ability to respond to a stress challenge is overwhelmed, physiologic reserve deteriorates. Fatigue, i.e., mental and/or physical exhaustion, ensues, reflecting an inability of the regulatory pathways to properly respond a given stress.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still allow a like or similar result without departing from the spirit and scope of the invention.

### Example 1

### Relationship of HRV Metrics to Level of Fatigue in Healthy Volunteer Subjects

### Materials and Methods

Selection Criteria.

The inventors recruited males between the ages of 18-35 years for inclusion in the current study. Potential subjects included healthy (asymptomatic) college students, ROTC cadet trainees, and recreational (non-varsity) athletes self-reporting as healthy and fit enough to enter basic military or first responder training, non-smoking, free of disease, and not taking any psychotropic medications or dietary supplements that would alter neural or metabolic function.

Subjects meeting the eligibility criteria were asked to read and sign an informed consent document approved by the Committee for the Protection of Human Subjects in Research of the University of Texas at San Antonio. This committee approved this study and provided oversight of human research procedures. Consented subjects completed a basic medical history screening form, a standard physical examination, and a clinical graded treadmill stress test with electrocardiogram to identify pre-existing heart conditions that could compromise safe participation and determine aerobic fitness level via indirect calorimetry (TrueMax 2400, ParvoMedics Sandy, Utah). Final selection of subjects was dependent upon normal clinical results and history as determined by a participating physician indicating eligibility for safe inclusion in the study. During the health screening session consented subjects were familiarized with all testing planned for the subsequent 48 hour duty protocol. All protocols were conducted in the Exercise Biochemistry and Metabolism Laboratory of the Department of Health and Kinesiology at The University of Texas at San Antonio.

Experimental Design.

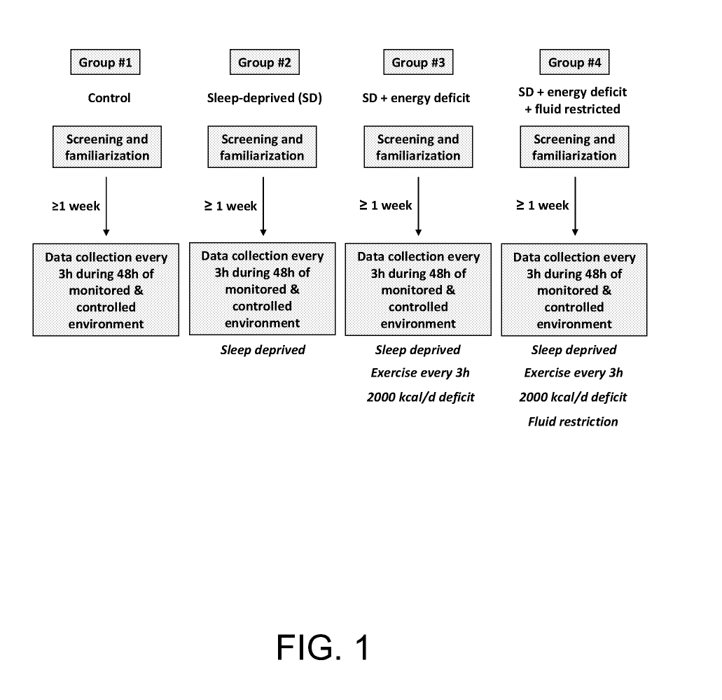

As shown in FIG. 1, all eligible subjects were randomly assigned to one of four experimental groups: 1) control; 2) sleep-deprived (SD); 3) SD+energy deficit; 4) SD+energy deficit+fluid restricted. All subjects participated in a single experimental group and each of the four experimental groups consisted of 14-16 subjects. The simulated 48 hour duty protocol took place at least one week following the health screening and familiarization session. The aim of the duty protocol was to increase mental and physical fatigue gradually and safely over the course of the 48 hours. In the present study, the inventors employed a modified version of a simulated 24 hour duty cycle protocol the inventors developed in which subjects experienced various combinations of sleep deprivation and caloric and/or fluid deficiency and were evaluated for cognitive performance and fatigue level every 3 hours.

Establishing Baseline Conditions for Feeding and Hydration.

For all groups, subjects reported to the laboratory at 0600 hours following an 8 hour fast that excluded caffeine or other stimulants. All subjects immediately received a standardized breakfast including 375 kcal of food and 1.2 L of water. For the purposes of analyzing data, the inventors define 0600 hours as Time=0. However, data for the 48 hour period were collected every 3 hours from 0900 hours on day one to 0600 hours on day three for eight data collection points each day (16 data collection points total). Every data collection point was 2 hours post-prandial and post-fluid ingestion.

### Group-Specific Details

Group 1. Control.

Group 1 subjects were allowed to sleep between the hours of 2200-0900 hours, although they were awoken at 0000 hours, 0300 hours and 0600 hours for approximately 1 hour to eat and drink, as well as to complete both the cognitive performance tests and the subjective fatigue survey. During non-sleep hours between 3 hour data collection periods, subjects maintained a controlled but normal daily schedule of very light activities (e.g., watching movies, studying or reading for enjoyment). Subjects were monitored throughout these between-data-collection periods and not allowed to sleep. Total dietary food and fluid intakes were controlled and provided at levels considered normal for the subject's age, weight, and daily activity level, allowing subjects to remain hydrated (i.e., total body water ˜60% of body weight).

Group 2. Sleep-Deprived.

Subjects were monitored throughout the 48 hour period and were not allowed to sleep. Schedules for food and water intake, as well as data collection, were the same as for Group 1. Activity levels between data collection periods were similar to those for Group 1.

Group 3. Sleep-Deprived+Energy Deficit.

Subjects were monitored throughout the 48 hour period and were not allowed to sleep. Schedules for food and water intake, as well as data collection, were the same as for Group 1. In addition, the subjects were required to exercise moderately for 1 hour immediately following each data collection point, beginning after the 0900 hours data collection. Group 3 subjects consumed half of their allotted water volume with their meal and half after the bout of exercise. Group 3 subjects performed 60 minutes of moderate exercise (brisk walk/march) immediately after their meal. Subjects performed ˜420 kcal of exercise during each session. The inventors estimated that the additional 15 exercise bouts led to an average caloric deficiency of 6300 kcal over the 48 hours. Daily energy expenditure and intake can vary widely depending on a person's operational tasks but elite combat personnel have demonstrated operational caloric deficits of 2500-4500 kcal/d (Aakwaag et al., 1978; Guezennec et al., 1994; Nindl et al., 2007). When not exercising, subjects' activity levels between data collection periods were similar to those of subjects in Group 1.

Group 4. Sleep-Deprived+Energy Deficit+Fluid Restricted.

Subjects were monitored throughout the 48 hour period and were not allowed to sleep. Schedules for food and water intake, as well as data collection, were the same as for Group 1. Group 4 exercise was the same as that for Group 3. Group 4 water intake at each 3 hour point was limited in order to dehydrate subjects gradually by approximately 5 kg (i.e., 5 L or 11% deficit in total body water) by the end of the 48 hour period as described below. A 5 L net water loss is predicted to be accompanied by a 5 kg body weight loss (−6% body weight) and an estimated 5% or 0.2 L loss of plasma volume by the end of the experiment as most of the net water loss is assumed to come from the intracellular and interstitial fluids (Nadel et al., 1980; Nose et al., 1983). It should be noted that Group 3 and 4 subjects performed their exercise bouts in mild environmental conditions within a range of 50-80° F. and below 70% relative humidity. When not exercising, subject's activity levels between data collections were similar to those of subjects in Group 1.

Feeding and Hydration.

Immediately after completing cognitive performance tests and Profile of Mood States survey (see below), subjects were fed and provided water at levels determined by their group, as described above. Food consisted of a small sandwich, raw vegetables and small cookies totaling 300 kcal/meal [50% carbohydrate (38 g), 30% fat (10 g), and 20% protein (15 g)]. This feeding schedule provided (including the standardized breakfast of 375 kcal) 5000 kcal over 48 h.

Groups 1 and 2 received 0.4 L of water with each meal for 7.2 L/48 hours (including the 1.2 L prior to data collection). Minimal water content of food was not considered in our estimates. Group 3 received 0.3 L of water with each meal and another 0.3 L after exercise for 10.2 L/48 hours. Group 4 received 0.4 L of water with each meal and no water after exercise (7.2 L/48 hours including 1.2 L prior to data collection) which was estimated to result in an approximate 3 L deficit by the end of the 48 hour duty protocol based on a predicted fluid requirement of 10 L/48 hours (Godek et al., 2005; Ruby et al., 2003).

Data Sampling.

Every 3 hours during the 48 hour period, subjects were weighed (in shorts and shirt, sans shoes) and then sat quietly for 20 minutes prior to subsequent data collection. Subjects were equipped with a commercial heart rate monitor (model RS810, Polar Electro, Kempele, Finland) that had previously been validated against laboratory ECG-based HRV measures by our group (data unpublished) and others (Radespiel-Tröger et al., 2003). Subjects were positioned supine for a 10 minute collection period followed immediately by a 10 minutes standing collection during which subjects maintained a 70° tilt leaning with head, shoulders, back, and elbows against a wall with feet 12 inches from the wall. The inventors did not record respiratory rate, but subjects breathed in time to a metronome set at a pace of 15 breaths per minutes (0.25 Hz) for the entire 20 minutes. Cooke et al. (1998) have shown previously that heart rate variability tracks respiratory frequencies during metronomic breathing at various frequencies. Individual heart beats were detected by the heart rate monitor and downloaded to computer for off-line analysis. All calculations of heart rate variability parameters were performed with commercially available data analysis software (WinCPRS, Absolute Aliens, Turku, Finland). Time- and frequency-domain components of HRV were assessed from each parameter average during the last 5 minutes of the 10 minutes data collection period (Table 1).

TABLE 1
Time And Frequency-Domain Components Of HRV

| Metric (units) | Definition | Interpretation |
|---|---|---|
| RRI_{avg} (ms) | Average absolute time between each | Associated with changes in vagal-cardiac |
| | R-wave during 20 min supine test | nerve activity |
| | period | |
| RRISD (ms) | Standard deviation of consecutive RRI | A combination of ANS factors |
| | | determining long-term heart rate |
| | | variability |
| RRIHF (ms²) | Power spectral density (integrative area | Changes are associated with changes in |
| | under 0.15-0.4 Hz) of the high | vagal-cardiac modulation |
| | frequency spectrum as derived from a | |
| | Fourier transform | |
| RRILF (ms²) | Power spectral density (integrative area | Changes are associated with, but not |
| | under 0.04-0.15 Hz) of the low | limited to, changes in both vagal- and |
| | frequency spectrum as derived from a | sympathetic-cardiac modulation |
| | Fourier transform | |

The inventors calculated standard time-domain statistics: average R-R interval (RRI_{avg}) and R-R interval standard deviation (RRISD). These time-domain statistics reflect both long- and short-term HRV mediated by both ANS and non-ANS sources. For representation of HRV in the frequency domain, RRI_{avg} were replotted using linear interpolation and resampled at 5 Hz. Data then were passed through a low-pass impulse response filter with a cutoff frequency of 0.5 Hz. Data sets were submitted to a Fourier transform with a Hanning window. The magnitude of RRI_{avg} oscillations were quantified by calculating the power spectral density for the ECG signal (total; 0.04-0.4 Hz). Signal areas were separated into high frequency (RRIHF; 0.15-0.4 Hz) and low frequency (RRILF; 0.04-0.15 Hz) bands (Task Force, 1996). Oscillations at the RRIHF represent modulation of HRV by parasympathetic efferent traffic, whereas oscillations at the RRILF represent modulation by a combination of factors, which include influences of both parasympathetic and sympathetic efferent traffic amongst other influences. The inventors report the RRI, RRISD, RRIHF, and RRILF for this investigation as changes in parasympathetic influence are of particular interest to fatigue (Jouanin et al., 2004). Following HRV testing, subjects completed the Profile of Mood States (POMS; McNair et al., 1971) survey and Stroop Color Conflict Test (Stroop tests; Frankenhaeuser and Johansson, 1976) of cognitive performance. Finally, mid-stream urine (˜125 ml) was collected for immediate determination by refractometry of urine specific gravity and implied hydration status.

Data and Statistical Analyses.

All statistical analyses were performed using R (version 2.9.0; cran.r-project.org). The nlme package (Pinheiro and Bates, 1996) for R was used to construct linear mixed-effects models to analyze changes in cognitive performance and HRV parameters with respect to changes in fatigue over the course of 48 hour. Our significance level was set a priori at p<0.05. Details of data/results/fits are provided in the legends accompanying results, figures, and tables.

### Results

### Descriptive Statistics for All Subjects

Descriptive statistics for the subjects at the start of the study are shown in Table 2. Anthropometric data, resting vital signs, and fitness levels were consistent with those of health, active males of comparable age (ACSM, 2000). Subjects in Group 1 self-reported 11.1±0.6 hours of cumulative sleep over the 48 hour protocol during the hours of 2200-0900 while subjects in sleep-deprived groups (2-4) did not sleep.

TABLE 2
Subject Characteristics For Fatigue Threshold Subjects
By Group Prior To The 48 Protocol

| | | | | Group 4 |
|---|---|---|---|---|
| | | Group 2 | Group 3 | SD + energy |
| | Group 1 | Sleep-deprived | SD + energy | deficit + fluid |
| | Control | (SD) | deficit | restricted |
| | n = 16 | n = 19 | n = 16 | n = 18 |
| Age (yr) | 21.3 ± 2.1 | 21.6 ± 2.9 | 20.8 ± 2.6 | 21.7 ± 4.6 |
| Weight (kg) | 78.0 ± 12.7 | 74.6 ± 10.0 | 81.7 ± 11.8 | 83.9 ± 11.7 |
| Height (cm) | 178.3 ± 0.1 | 177.3 ± 0.1 | 178.4 ± 0.1 | 179.7 ± 0.1 |
| Body mass index | 24.5 ± 3.2 | 23.9 ± 4.0 | 25.7 ± 3.8 | 26.2 ± 4.7 |
| Body fat (%) | 16.0 ± 5.8 | 16.2 ± 7.5 | 19.3 ± 7.2 | 19.6 ± 8.7 |
| VO2 peak | 45.2 ± 6.4 | 46.7 ± 6.9 | 44.0 ± 7.8 | 44.2 ± 5.9 |
| (ml O₂ * kg⁻¹ * min⁻¹) | | | | |
| Systolic blood | 121.8 ± 8.0 | 120.5 ± 8.6 | 122.8 ± 9.1 | 119.0 ± 9.6 |
| pressure (mmHg, | | | | |
| seated rest) | | | | |
| Diastolic blood | 77.1 ± 5.4 | 77.0 ± 5.8 | 73.6 ± 7.1 | 81.9 ± 6.3 |
| pressure (mmHg, | | | | |
| seated rest) | | | | |
| Heart rate (bpm, | 66.6 ± 11.2 | 66.1 ± 8.8 | 71.0 ± 14.2 | 84.9 ± 9.7* |
| seated rest) | | | | |
| Urine specific gravity | 1.008 ± 0.00† | 1.018 ± 0.01† | 1.009 ± 0.01 | 1.014 ± 0.01†† |

Data are presented as mean ± SD.
*P < 0.05 vs. groups 1-3,
†P < 0.05 vs. group 3,
††P < 0.05 vs. group 1

During the 48 hour protocol, Group 4 average body weight decreased by 1.4 kg (2%) (p<0.05, data not shown), while remaining constant for all remaining groups. Urine specific gravity (USG) increased significantly over the 48 hour protocol only in Group 4 (1.014±0.01 at 3 hours versus 1.036±0.004 at 48 hours; p<0.05).

### Widely Varying Levels of Fatigue Among Groups and Subjects

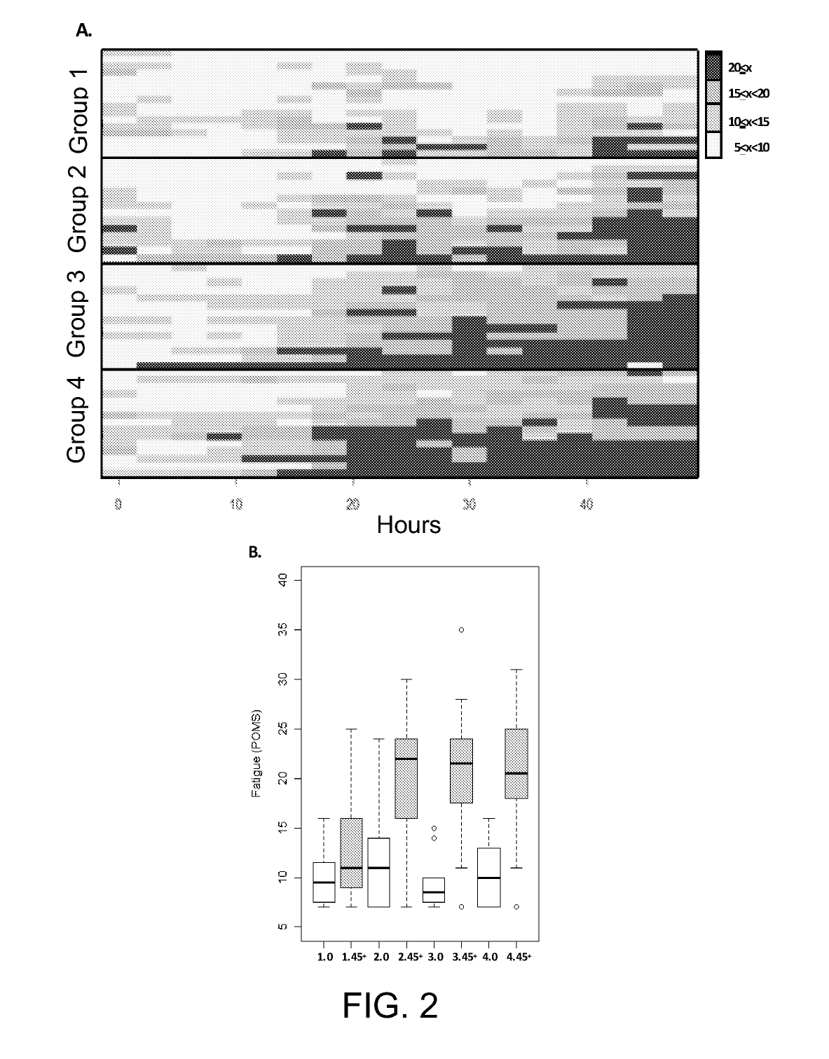

In this study, each subject belonged to one of four groups with each group following a different protocol with the intent of inducing varying levels of fatigue among the subjects. As illustrated in FIG. 2, the different protocols led to widely varying levels of fatigue among groups and subjects, even though the starting levels of fatigue were similar for all subjects (Kruskal-Wallis test for data at Time=0 hours, p>0.05).

In FIG. 2A, POMS Fatigue levels are shown for each subject as a function of time. Data from each subject are displayed as a single row with POMS Fatigue level encoded by the greyscale shown a the right.

In FIG. 2B, POMS Fatigue levels are shown as boxplots with data separated by group and by time. For the boxplots, the dark central line indicates the position of the median and the lower and upper bounds of the rectangles denote the positions of the first and third quartiles, respectively; open circles denote individual data points at the extremes. The differences in POMS Fatigue levels across time, but within Group, are significant except for Group 1.

Individual POMS Fatigue levels ranged from 7 to 35, with values varying more between subjects in different groups than between subjects within a group. For example, more than 60% of the POMS Fatigue scores were less than or equal to 10 for subjects in Group 1, while less than 25% of the POMS Fatigue scores were <10 for Group 4. Because the object of this study was to investigate physiological changes in response to significant increases in fatigue, the inventors limited further analysis to those individuals whose POMS Fatigue levels exceeded 20 in at least one time point. Importantly, once included, all of a subject's data across the entire 48 hour period were used, not just the measurements associated with a POMS Fatigue level greater than 20. Thus, some control subjects (Group 1) did, in fact, reach a level of subjective fatigue during the study to justify inclusion in the subsequent modeling. The limited data set resulting from the application of this threshold included 41 of the original 69 participants. Of the 41 subjects in the reduced data set, 37 completed the entire study. The number of participants from each group included in the reduced data set is as follows: 4 (Group 1), 11 (Group 2), 14 (Group 3) and 12 (Group 4).

### Cognitive Performance Decreased as Fatigue Increased

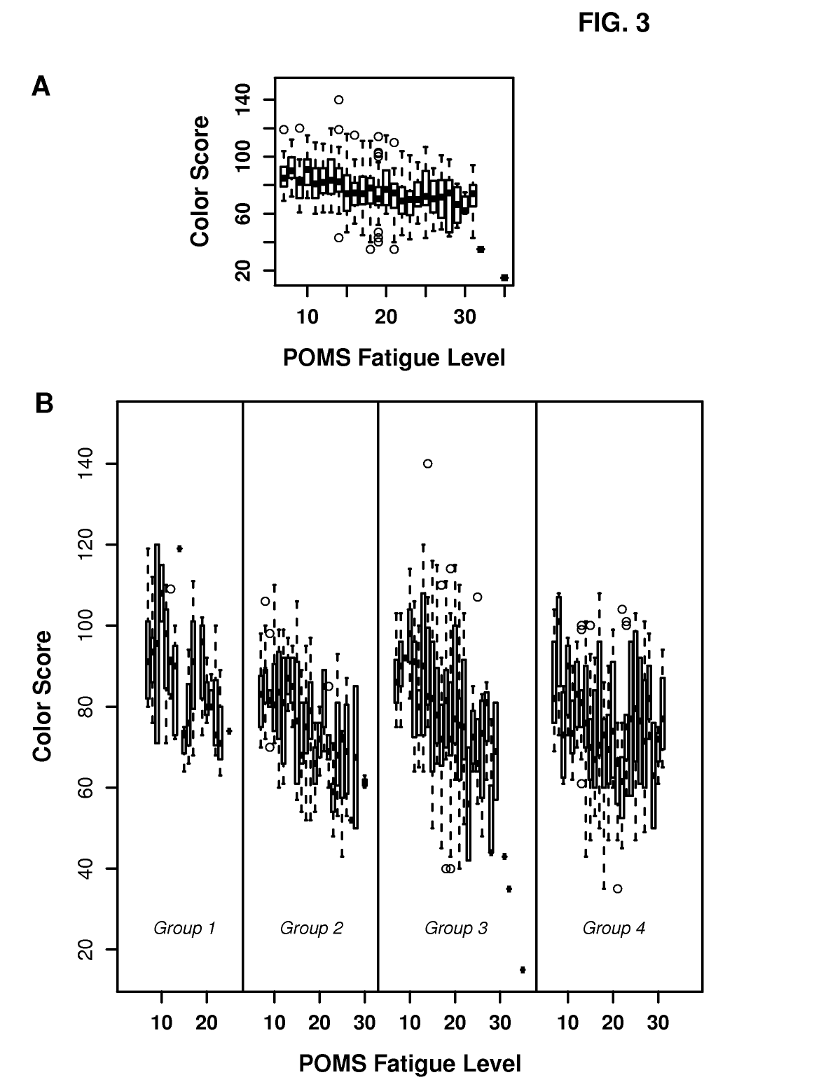

Increased levels of fatigue were associated with lower scores on cognitive performance tests. In this study, the inventors used the Stroop Color Conflict Test (Stroop tests) to quantify cognitive performance. The Stroop tests comprise three separate tests: Color, Word and Color-word. As shown in FIG. 3, the relationship between cognitive performance and fatigue was apparent at multiple levels of grouping for the data, i.e. the population level and the Group level. In FIG. 3, the scores on the Stroop Color test are plotted against POMS Fatigue level for two different levels of grouping of the data: (A) population level and (B) Group level. Data are shown in boxplots, with a dark central line indicating the position of the median and the lower and upper bounds of the rectangles denoting the positions of the first and third quartiles, respectively; open circles denote individual data points at the extremes.

In FIG. 3, a non-zero slope suggests a fundamental relationship between scores on the Stroop test and the level of fatigue. As observed for Stroop Color test scores in FIG. 3, non-zero slopes seem appropriate for the population level, as well as for all levels of Group. To test the significance of the observed decrease in cognitive performance, the inventors used linear mixed-effects (LME) modeling, an approach that can distinguish effects attributable to the population, or subsets of the population, (fixed effects) from effects specific to a particular subject (random effects). Scores for each Stroop test were modeled separately using the general form of the LME equation given in Equation 1.

General Form of the LME Equation for Fitting Stroop Test Scores StroopXᵢⱼ=(β₀+γ₀₂G₂ᵢ+γ₀₃G₃ᵢ+γ₀₄G₄ᵢ+b₀ᵢ)+(β₁+γ₁₂G₂ᵢ+γ₁₃G₃ᵢ+γ₁₄G₄ᵢ+b₁ᵢ)*Fᵢⱼ+εᵢⱼ Equation 1.

In Equation 1, StroopXᵢⱼ is the jth result for subject i on Stroop test X, where X=Color, Word or Color-word; Gₖᵢ are binary variables with value of 1 when subject i belongs to Group k and value of 0 otherwise; β₀ and β₁ are the average intercept and average slope for the subjects in Group 1, respectively; γ₀ₖ is the average difference in the intercept between subjects in Group k and subjects in Group 1; γ₁ₖ is the average difference in the slope between subjects in Group k and subjects in Group 1; b₀ᵢ and b₁ᵢ are the random-effects terms for subject i for the intercept and slope, resp., assumed independent for different i; Fᵢⱼ is the jth “Fatigue” score for subject i, determined using the POMS survey; and εᵢⱼ is the within-group error, assumed independent for different i,j and independent of the random effects.

After fitting the results to the most general LME model (Equation 1), the inventors sequentially removed the least significant term (as determined by ANOVA analysis) and determined whether this reduced model was at least as good as the previous, i.e. more general, model. This process was continued until either the more general model was more appropriate than the reduced model or only a single term remained. Significant fixed-effects coefficients from the best-fit LME models are reported in Table 3.

TABLE 3
Linear Mixed-Effects Modeling of Cognitive Performance Tests

| | |
|---|---|
| β₀ | β₁ |

All Data

| | | |
|---|---|---|
| Color Test | 75.8 ± 1.9* | −1.15 ± 0.10* |
| Word Test | 109.4 ± 2.1* | −1.01 ± 0.11* |
| Color-word Test | 55.7 ± 1.3* | −0.44 ± 0.08* |

Threshold Data

| | | |
|---|---|---|
| Color Test | 76.9 ± 2.0* | −1.13 ± 0.11* |
| Word Test | 109.6 ± 2.4* | −0.91 ± 0.12* |
| Color-word Test | 55.4 ± 1.4* | −0.42 ± 0.09* |

Fixed-effects coefficients are reported for the final LME model describing the relationship between each Stroop test and POMS “Fatigue” score.
*Indicates that the term is significantly different from 0.

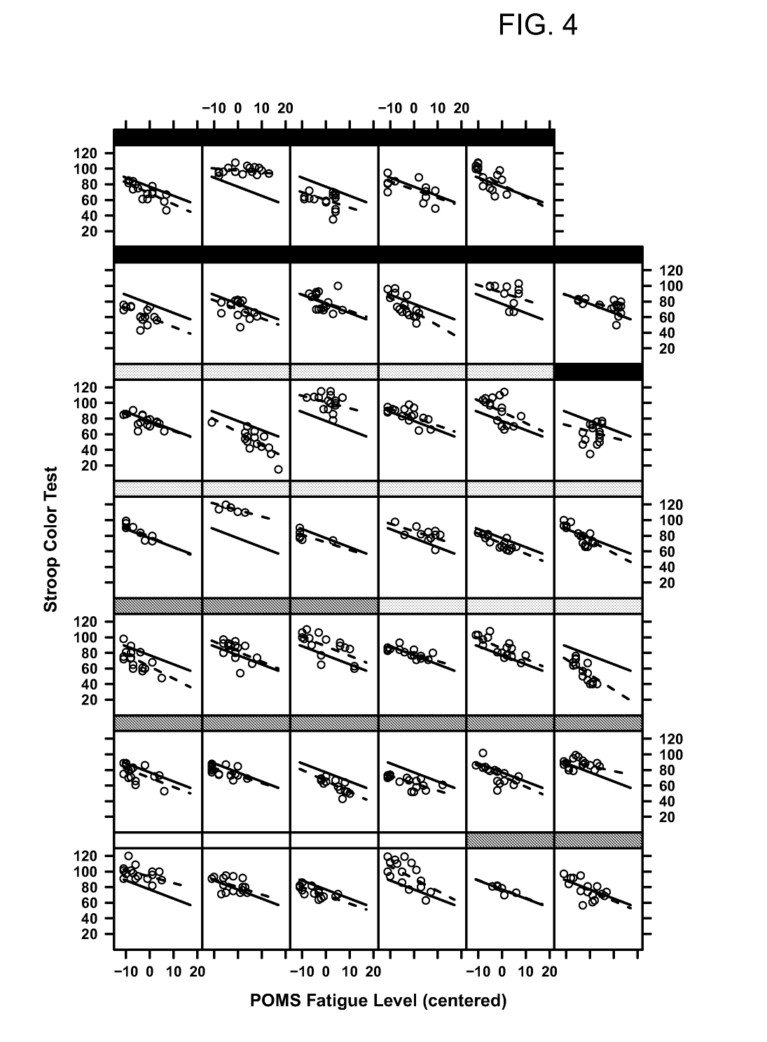

The LME results apply to all levels of grouping for the data: population, Group and subject. An example of the best fit LME model is shown graphically in FIG. 4. Here, scores on the Stroop Color test are plotted against POMS Fatigue level separately for all subjects. In each panel, open circles show the actual data for the subject. The solid line shows the fit for the population and the dotted line shows the fit for the subject.

### Heart Rate Variability Parameters

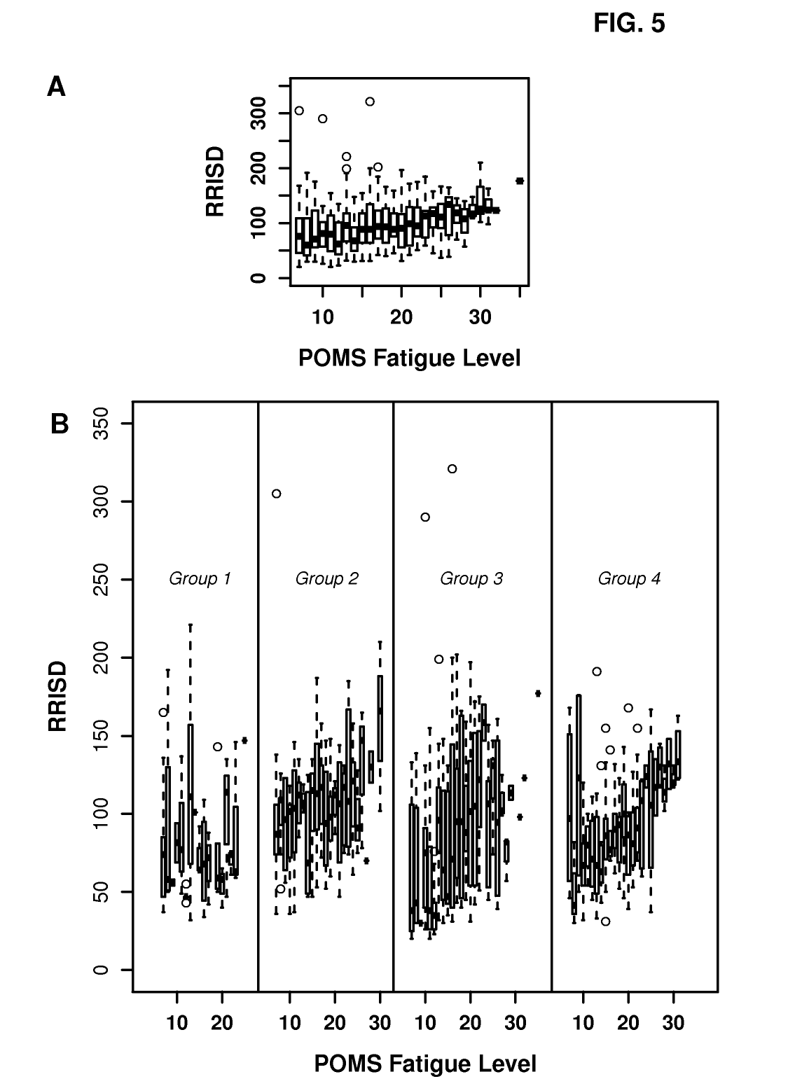

For each of our HRV parameters of interest, data were analyzed with respect to POMS fatigue level. As with Stroop test results above, data for each HRV parameter were initially plotted at two levels of grouping: 1) the population level, i.e. one plot using all data from all subjects and 2) the Group level, i.e. four separate plots, each using data from only a single group. A representative example of this procedure is provided for RRISD in FIG. 5.

In FIG. 5, a non-zero slope suggests a fundamental relationship between the HRV parameter and the level of fatigue. For the HRV parameter, RRISD, non-zero slopes seem appropriate for the population level, as well as for all levels of Group. In FIG. 5, the HRV metric RRISD is plotted against POMS Fatigue level for two different levels of grouping of the data: (A) population level and (B) Group level. Data are shown in boxplots, with a dark central line indicating the position of the median and the lower and upper bounds of the rectangles denoting the positions of the first and third quartiles, respectively; open circles denote individual data points at the extremes.

### Linear Mixed-Effects Models of Heart Rate Variability Parameters

To investigate rigorously the relationship between HRV parameters and a subject's self-reported level of fatigue, the inventors used linear mixed-effects (LME) modeling. For this analysis, the inventors constructed a separate LME model for each of the four HRV parameters of interest: 1) RRI, 2) RRISD, 3) RRIHF and 4) RRILF. To select the most appropriate model for each HRV parameter, the inventors began with the general LME model shown in Equation 2.

General Form of the LME Equation for Fitting HRV Parameters HRVXᵢⱼ=(β₀+γ₀₂G₂ᵢ+γ₀₃G₃ᵢ+γ₀₄G₄ᵢ+b₀ᵢ)+(β₁+γ₁₂G₂ᵢ+γ₁₃G₃ᵢ+γ₁₄G₄ᵢ+b₁ᵢ)*Fᵢⱼ+εᵢⱼ Equation 2.

In Equation 2, HRVXᵤ is the jth observation for subject i of HRV parameter X (X=RRI, RRISD, RRILF, or RRIHF); Gₖᵢ are binary variables with value of 1 when subject i belongs to Group k and value of 0 otherwise; β₀ and β₁ are average intercept and average slope for subjects in Group 1, respectively; γ₀ₖ is the average difference in the intercept between Group k and Group 1; γ₁ₖ is the average difference in the slope between Group k and Group 1; b₀ᵢ and b₁ᵢ are the random-effects terms for subject i for the intercept and slope, resp., assumed independent for different i; Fᵢⱼ is the jth “Fatigue” score for subject i, determined using the POMS survey; and εᵢⱼ is the within-group error, assumed independent for different i,j and independent of the random effects.

After fitting the results to the most general LME model (Equation 2), the inventors sequentially removed the least significant term (as determined by ANOVA analysis) and determined whether this reduced model was at least as good as the previous, i.e. more general, model. This process was continued until either the more general model was more appropriate than the reduced model or only a single term remained. Significant fixed-effects coefficients from the best-fit LME models are reported in Table 4.

TABLE 4
Linear Mixed-Effects Modeling of HRV Parameters.

| | | | | | | | |
|---|---|---|---|---|---|---|---|
| β₀ | γ₀₂ | γ₀₃ | γ₀₄ | β₁ | γ₁₂ | γ₁₃ | γ₁₄ |

All Data.

| Supine | | | | | | | | |
|---|---|---|---|---|---|---|---|---|
| RRI | 1060 ± 35* | −1 ± 46 | −117 ± 47^{†} | −122 ± 46^{†} | 2.4 ± 2.2 | 7.3 ± 2.8^{†} | 6.5 ± 2.8^{†} | 8.6 ± 2.8^{†} |
| DEV | 98.4 ± 3.8* | NS | | | −0.2 ± 0.6 | 1.8 ± 0.8^{†} | 3.0 ± 0.8^{†} | 3.0 ± 0.8^{†} |
| log₁₀(RRIHF) | 3.04 ± 0.05* | NS | | | −0.007 ± 0.008 | 0.023 ± 0.010^{†} | 0.055 ± 0.011^{†} | 0.040 ± 0.011^{†} |
| log₁₀(RRILF) | 3.27 ± 0.04* | NS | | | 0.014 ± 0.007* | 0.012 ± 0.009 | 0.037 ± 0.009^{†} | 0.025 ± 0.009 |
| Standing | | | | | | | | |

| | | | | | | |
|---|---|---|---|---|---|---|
| RRI | 775 ± 24* | −17 ± 31 | −104 ± 33^{†} | −107 ± 32^{†} | 3.87 ± 0.66* | NS |
| DEV | 93.2 ± 5.1* | −7.0 ± 5.8 | −28.2 ± 6.3^{†} | −27.0 ± 6.2^{†} | 1.54 ± 0.22* | NS |

| | | | | | | |
|---|---|---|---|---|---|---|
| log₁₀(RRIHF) | 2.21 ± 0.04* | NS | −0.009 ± 0.006 | 0.018 ± 0.007^{†} | 0.025 ± 0.007^{†} | 0.028 ± 0.007^{†} |
| log₁₀(RRILF) | 3.13 ± 0.04* | NS | 0.007 ± 0.004 | 0.006 ± 0.005 | 0.012 ± 0.005^{†} | 0.019 ± 0.005^{†} |

Threshold Data.

| Supine | | | | | | |
|---|---|---|---|---|---|---|
| RRI | 982 ± 20* | NS | 9.1 ± 1.1* | NS | | |
| DEV | 97.8 ± 4.5* | NS | 2.1 ± 0.4* | NS | | |
| log₁₀(RRIHF) | 3.02 ± 0.05* | NS | 0.001 ± 0.013 | 0.016 ± 0.016 | 0.050 ± 0.015^{†} | 0.027 ± 0.016 |
| log₁₀(RRILF) | 3.28 ± 0.05* | NS | 0.022 ± 0.011* | 0.002 ± 0.013 | 0.031 ± 0.013^{†} | 0.011 ± 0.013 |
| Standing | | | | | | |

| RRI | 705 ± 16* | NS | | | 3.89 ± 0.71* | NS |
|---|---|---|---|---|---|---|
| DEV | 83.7 ± 8.1* | 1.5 ± 8.6 | −21.4 ± 8.6^{†} | −19.1 ± 8.8^{†} | 1.54 ± 0.25* | NS |
| log₁₀(RRIHF) | 2.19 ± 0.06* | NS | | | 0.012 ± 0.003* | NS |
| log₁₀(RRILF) | 2.93 ± 0.13* | 0.36 ± 0.16^{†} | −0.01 ± 0.15 | 0.19 ± 0.15 | 0.018 ± 0.002* | NS |

Fixed-effects coefficients are reported for the final LME model describing the relationship between each HRV parameter and POMS “Fatigue” score.
*Indicates that the term is significantly different from 0.
^{†}Indicates that the difference is significant with respect to the corresponding term for Group 1 (βX for γXₖ, where X = 0.1 and k indicates group number).

As indicated in Table 4, the inventors modeled the log₁₀ transform of data for RRILF and RRIHF. The decision to use log₁₀ transforms for these parameters was made after observing a wedge-shaped distribution of residuals (εᵢⱼ's), when plotted against fitted values for models constructed using raw values of RRIHF and RRILF. With log₁₀ transformed data, the residuals appeared normally distributed with respect to the fitted values. Separate analysis of the distribution of values for RRIHF and RRILF suggested a log-normal, rather than normal distribution (data not shown).

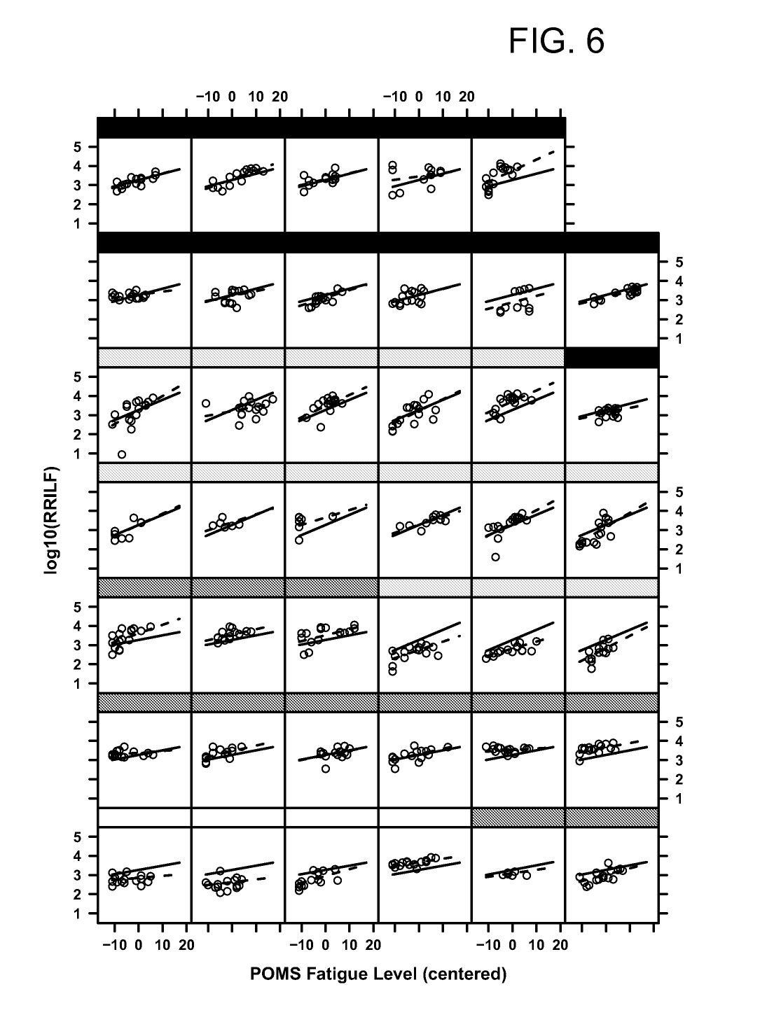

The LME results apply to all levels of grouping for the data: population, Group and subject. An example of the best fit LME model is shown graphically in FIG. 6. In FIG. 6 individual subject's RRILF values are plotted against the POMS Fatigue level. In each panel, the open circles show the actual data for the subject. The solid line shows the fit for the population and the dotted line shows the fit specific to the subject.

### Example 2

### Methods to Quantify Levels of Fatigue

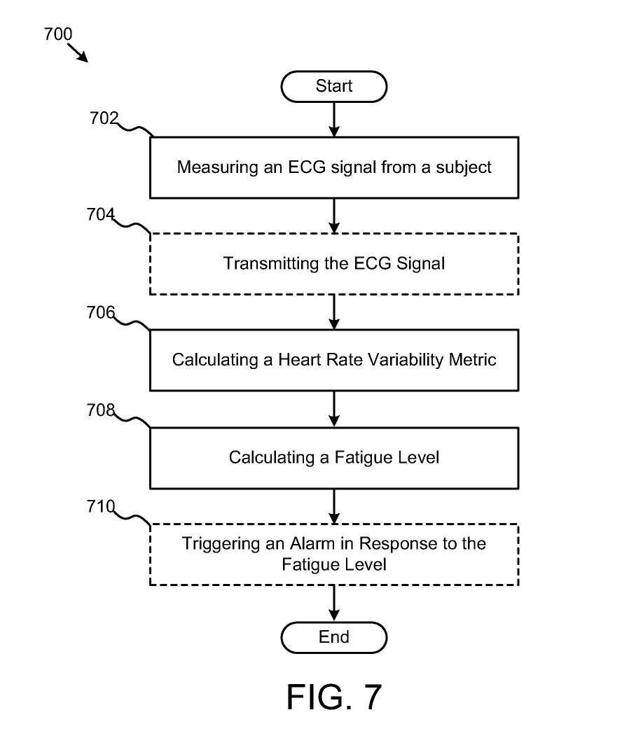

Methods are disclosed for quantifying fatigue of a subject. FIG. 7 shows a flow chart that demonstrates certain embodiments of a method 700 for quantifying fatigue of a subject. In certain embodiments, the method 700 may include measuring 702 an ECG signal from a subject. The ECG signal reflects electrical changes on the skin created in response to the signaling in the heart muscle that controls each heartbeat. The ECG signal may be measured using two or more electrodes or pads. This may be done using any method known to those of ordinary skill in the art. Embodiments for measuring an ECG signal will be discussed later in this section. In certain embodiments, the ECG signal is converted from an analog signal to a digital signal using a digital-to-analog converter. This conversion of the ECG signal from analog-to-digital allows for more efficient and accurate processing of the ECG signal.

In certain embodiments, after measuring 702 the ECG signal, the ECG signal may be transmitted 704 to a processing device. The transmission 704 of the ECG signal may be a wireless transmission or a wired transmission. In certain embodiments, the device used to measure the ECG signal and the processing device are coupled together, and the transmission 704 is wired. In other embodiments, the two devices are separate, and the transmission 704 is wireless. The transmission 704 of the ECG signal allows for further processing of the ECG signal by a processing device. In certain embodiments, the transmission 704 of the ECG signal may include encrypting the ECG signal. In certain embodiments, the transmission 704 of the ECG signal may include encoding the ECG signal. For example, an encoded ECG signal may only be decoded by a processing device enabled to decode a specific ECG signal. One having skill in the art can recognize several techniques for transmitting 704 and encrypting an analog or digital signal to a processing device, and this process is not discussed in detail in this disclosure.

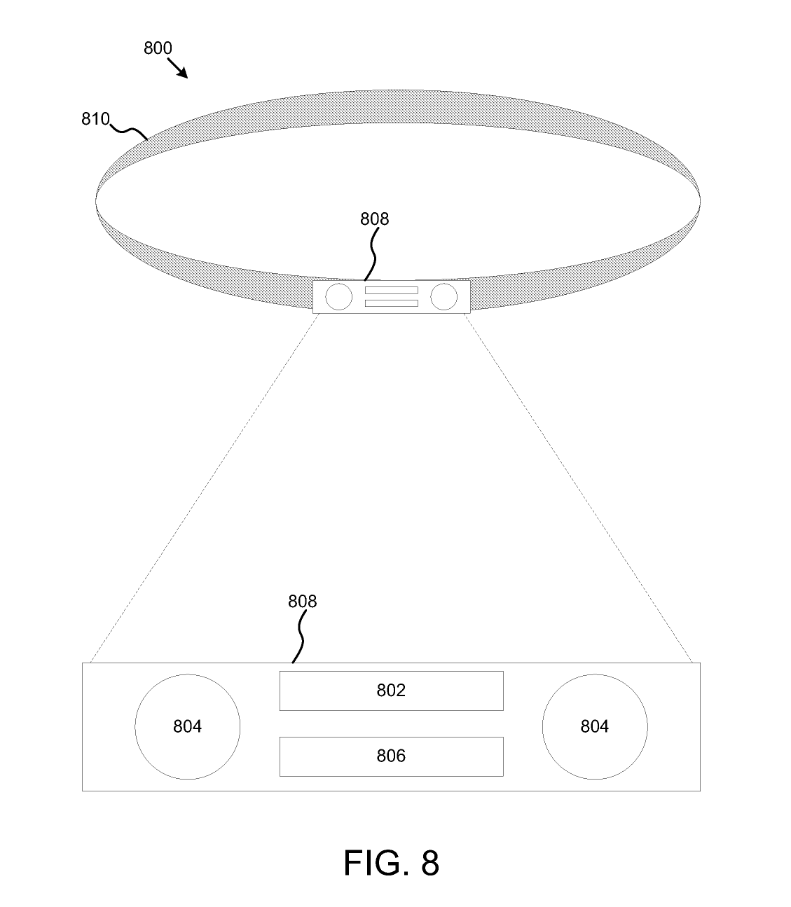

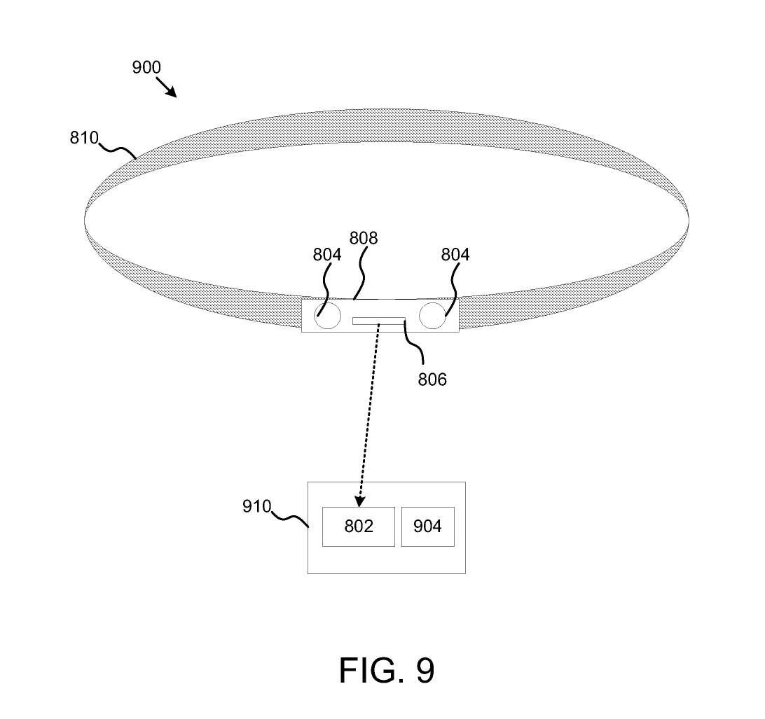

In certain embodiments, the method 700 further includes calculating 706, with a processing device, an HRV metric in response to the ECG signal. Embodiments of the processing device will be discussed in greater detail with respect to FIG. 8 and FIG. 9. Four HRV metrics—RRI, RRISD, RRILF, or RRIHF—are disclosed in Table 1. One or more of these HRV metrics may be calculated 706 in response to the ECG signal.

In certain embodiments, calculating 706 an HRV metric includes determining the RRI over a period of time. In certain embodiments, this period of time is 30 seconds to 15 minutes. In a preferred embodiment, this period of time is 10 minutes. Similarly, in certain embodiments, calculating 706 an HRV metric may also include determining the RRISD over a period of time. In certain embodiments, this period of time is 30 seconds to 15 minutes. In a preferred embodiment, this period of time is 10 minutes.

In certain embodiments, calculating 706 the HRV metric includes calculating the power spectral density of the ECG signal. The power spectral density describes how the power of a signal is distributed with frequency. One of ordinary skill in the art of signal processing can recognize techniques for calculating the power spectral density of a signal. Calculating the power spectral density may include filtering the ECG signal with a low-pass impulse response filter. An impulse response filter is a digital filter well-known in the art of signal processing. Filtering the ECG signal produces a filtered ECG signal. In other embodiments, the ECG signal may be filter with an analog low-pass filter. In certain embodiments, the low-pass filter used to calculate the power spectral density may have a cut-off frequency of 0.5 Hz. In other words, the low-pass impulse response filter will filter out frequencies higher than 0.5 Hz.

Calculating the power spectral density further includes performing a Fourier transform on the filtered ECG signal to form a processed ECG signal. A Fourier transform is a well-known mathematical computation in the art of signal processing. In certain embodiments, the Fourier transform may also include a Hanning window. The Hanning window—also referred to as a Hann window—is a windowing function known in the art of signal processing. One of skill in the art may recognize other window functions used to process the frequency domain signal.

In certain embodiments, calculating 706 the HRV metric comprises calculating the power spectral density of the ECG signal across a frequency range from 0.04 Hz to 0.15 Hz. As described in Table 1, this HRV metric is the RRILF metric. Similarly, in certain embodiments, calculating 706 the HRV metric comprises calculating the power spectral density of the ECG signal across a frequency range from 0.15 Hz to 0.4 Hz. As described in Table 1, this HRV metric is the RRIHF metric. After filtering, the frequency range of the ECG signal ranges from 0 Hz to approximately 0.5 Hz. The RRILF metric thus analyzes the power spectral density of the lower frequency range of the ECG spectrum, and the RRIHF metric analyzes the power spectral density of the higher range of the ECG spectrum.

In certain embodiments, the method 700 further includes calculating 708, with a processing device, a fatigue level in response to the HRV metric. The inventors used linear mixed-effects (LME) modeling to demonstrate linear correlations between fatigue level and a given HRV metric. For example, with respect to FIG. 6, inventors note that as the fatigue level of a subject increases, the RRILF of the an individual increases. Inventors determined a similar correlation for RRIHF, RRI, and RRISD. From the LME fits, the inventors were able to obtain the best-fit coefficients, i.e. slope and intercept, describing the relationship between the HRV metric (response; dependent variable) and fatigue level (co-variate; independent variable) at three levels of detail: 1) the population, 2) the group and 3) the individual. The values for the LME coefficients for four HRV metrics (RRI, DEV, RRIHF and RRILF) are shown in Table 4 at the level of the population and/or the Group. While not shown, best-fit LME coefficients were also derived at the level of the individual.

As presented in Example 1 of this application, HRV metrics were modeled as a function of fatigue level. This approach was taken, because there has not previously been a demonstration of a linear correlation between HRV metrics and fatigue level. However, for the described methods, apparatuses, and systems, the processing device would be capable of calculating fatigue level (response; dependent variable) from the HRV metric (covariate; independent variable). Specifically, the processing device must subtract the appropriate intercept term from the measured HRV metric and then divide the result by the appropriate slope term. That is, as written for Equation 2/Table 4 of this application, we have described the relationship between fatigue level and HRV metric as follows: HRV_{X}=m*Fatigue+b, whereas the processing device would be configured to calculate fatigue level as follows: Fatigue=(HRV_{X}−b)/m. The values of b (intercept) and m (slope) can come from several sources.

In certain embodiments, values of b and m could be taken directly from Table 4, using group-specific correction factors when appropriate. The values in Table 4 represent the response expected for the general population and are appropriate as a first approximation for all people. In other embodiments, specific values of slope and intercept may be calculated for each user. Obtaining the user-specific values of slope and intercept requires having the user complete a fatigue-inducing protocol similar to those described in Example 1/FIG. 1. During this protocol, ECG signals would be measured and the values for all HRV metrics derived using methods and devices similar to those described in Example 1 and FIGS. 7, 8 & 9. Slope and intercept terms for the user would be calculated from the data using methods known to one skilled in the art. For example, the collected data might be added to the data set collected in Example 1 and a new LME model fit, in the process establishing the best fit coefficients for the particular user.

In certain embodiments, the method 700 further includes triggering 710 an alarm in response to the fatigue level. For example, if an individual's fatigue is calculated 708 to be at an unsafe level, an alarm may be triggered 710 to warn the individual or other to take corrective action. In certain embodiments, the method 700 includes triggering 710 the alarm based on one more use specific levels of fatigue. In certain embodiments, the specific level of fatigue is user selectable. The triggered 710 alarm may be useful to indicate that the subject needs rest, food, or hydration. Such monitoring of quantified fatigue levels could be useful in many applications including monitoring the fatigue of doctors, athletes, airplane pilots, factor workers, construction workers, or anyone operating heavy machinery.

### Example 3

### Devices to Measure Cognitive Performance-Relevant Levels of Fatigue in Real Time

FIG. 8 and FIG. 9 illustrate embodiments of an apparatus 800 and a system 900 for quantifying the fatigue level of a subject. In certain embodiments, the apparatus 800 or the system 900 may be used to perform certain embodiments of the method 700. Fundamentally, embodiments of the apparatus 800 or the system 900 will measure heart-rate variability parameters, which are derived from continuous measurements of electrical activity in the heart, and make use of linear relationships to provide the user with a quantitative measurement of fatigue level and/or cognitive performance capacity.

In general, heart-rate variability (HRV) parameters are believed to be reliable surrogate measures of activity in the autonomic nervous system (ANS), a system that is known to play a central role in the physiological response to increases in fatigue. The inventors have recently used mathematical models to identify statistically significant relationships between subjective fatigue levels and several heart rate variability (HRV) parameters. Moreover, the inventors have demonstrated that increases in fatigue level were accompanied by statistically significant declines in cognitive performance. Embodiments of the apparatus 800 or the system 900 may be used as a diagnostic instrument to measure HRV parameters as indicators of fast-reactive autonomic regulation, making possible the identification of early physiologic alterations accompanying elevated fatigue levels and/or impaired cognitive performance.

The key physiological principle is the relationship between the autonomic nervous system and fatigue level. Because direct measurements of ANS activity are not practical, embodiments of the apparatus 800 or the system 900 instead rely on measurements of HRV parameters to infer ANS activity. HRV parameters are derived from readily measurable cardiac electrical signals, and embodiments of the apparatus 800 or the system 900 may be used for measuring autonomic functional capacity to identify the “risk” of an individual succumbing to stress (i.e., inability of body to respond or accommodate a level of stress) and dictate appropriate intervention strategies prior to cognitive performance decrements. Embodiments of the apparatus 800 or the system 900 may be used to quantify an individual's absolute level of fatigue or to measure relative changes in an individual's level of fatigue. Moreover, embodiments of the apparatus 800 or the system 900 may be used for monitoring individuals whose work requires activities particularly susceptible to fatigue, such as doctors, airplane pilots, truck drivers and military drone operators. Embodiments of the apparatus 800 or the system 900 may also be used to optimize demanding training regimens, such as those for elite athletes, military recruits and first responders.

FIG. 8 illustrates one embodiment of an apparatus 800 for quantifying fatigue. In certain embodiments, the apparatus 800 includes two or more ECG measuring pads 804 configured to measure an ECG signal from a subject. In a preferred embodiment, the apparatus 800 has two measuring pads 804. The measuring pads 804 may include electrical leads or other like instruments to measure the electric field associated with the contraction of the heart muscle. The measuring pads 804 may be configured to be positioned in contact with a surface of the subject.

The apparatus may also include a processing device 802. In certain embodiments, the processing device may be a CPU. The CPU 802 may be a general purpose CPU or microprocessor. The present embodiments are not restricted by the architecture of the CPU 802, so long as the CPU 802 supports the operations as described herein. The CPU 802 may execute the various logical instructions according to the present embodiments. For example, the CPU 802 may execute machine-level instructions according to the exemplary operations described below with reference to FIG. 7. The processing device 802 is not limited to a CPU. Moreover, the present embodiments may be implemented on application specific integrated circuits (ASIC) or very large scale integrated (VLSI) circuits. In fact, persons of ordinary skill in the art may utilize any number of suitable structures capable of executing logical operations according to the described embodiments. The apparatus 800 may even include one or more processing devices 802. The processing device 802 may configured to calculate 706 an HRV metric and calculate a fatigue level 708. In certain embodiments, the processing device 802 may be located in a piece of hardware distinct from the one containing the two measuring pads 804.

In certain embodiments, the apparatus 800 may include a transmitting device 806 to transmit the measured ECG signal to the processing device 802. As described above, the transmitting device 806, may transmit the measured ECG signal either wirelessly or through a wire.

In certain embodiments, the ECG measuring pad 804 and the processing device 802 are comprised in a strap or pad 808. The strap or pad 808 maybe attached to the subject whose fatigue is being quantified. In some embodiments, the strap or pad 808 may be affixed to the subject with a chest strap 810. Thus, in certain embodiments, the apparatus 800 can be worn around the chest of a subject.

In certain embodiments, the apparatus 800 may also include an alarm (not shown) configured to trigger 710 in response to a fatigue level.

FIG. 9 illustrates a system 900 for quantifying fatigue of a subject. As described above with respect to FIG. 8, the system 900 may include two or more ECG measuring pads 804 configured to measure the ECG signal from a subject. The system 900 may also include processing devices 802. In certain embodiments, the processing device 802 may be configured to calculate 706 a Heart Rate Variability (HRV) metric in response to the ECG signal and calculate 708 a fatigue level in response to the HRV metric. The system 900 may further include a transmitting device 806.

The transmitting device 806 may be configured to transmit 704 the ECG signal to the processing device 802. As depicted in FIG. 9, in certain embodiments, the transmitting device may wirelessly transmit 704 to a processing device 802.

In certain embodiments of system 900, the two or more ECG measuring pads 804 and the transmitting device 806 are comprised in a first strap or pad 808. The first strap or pad 808 may be attached to the subject. In certain embodiments, the first strap or pad 808 may be attached to the subject with chest strap 810. In certain embodiments, the processing device 802 may be comprised in a second strap or pad 910. The second strap or pad 910 may be physically separate from the chest region of the subject. For example, the second strap or pad 910 may be affixed the wrist of the subject—similar to a wrist watch. The second strap or pad 910 may be carried in the pocket or clipped to the subject—similar to a MP3 music player. The second strap or pad 910 may be affixed to the shoe or other part of the subject.

In other embodiments, the processing device 802, may not be attached to the subject at all, and rather be incorporated within a handheld device or personal computer monitored (not shown). Such personal computing devices include cell phones, PDAs, iPADs, laptops, and personal computers.

In other embodiments, the processing device 802 may be incorporated in a receiver (not shown). The receiver may transmissions 704 of an ECG signal from one or more subjects. In certain embodiments, the processing device 802 may process the ECG signals from one or more subjects in a centralized location. For example, in such embodiments, a coach, a commander, a manager, or other person may monitor several subjects at once from one centralized location.

The system 900 may further include an alarm 904 configured to trigger in response to the fatigue level. In certain embodiments, the alarm is contained within the second strap or pad 910.

All of the methods disclosed and claimed herein can be made and executed without undue experimentation in light of the present disclosure. While the methods of this invention have been described in terms of preferred embodiments, it will be apparent to those of skill in the art that variations may be applied to the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
Aakwaag et al., Eur. J. Appl. Physiol., 39: 283-91, 2004.
American College of Sports Medicine, ACSM's guidelines for exercise testing and prescription, Philadelphia, Lippencott, Williams, and Wilkins, 2000.
Angus and Heslegrave, Behav. Res. Methods Instrum. Comput., 17:55-67, 1985.
Boonstra et al., Cell Mol. Life Sci., 64: 934-946, 2007.
Chuah L Y, Chee M W. Cholinergic augmentation modulates visual task performance in sleep-deprived young adults. J Neurosci 29: 11369-11377, 2008.
Cooke et al., Am. J. Physiol., 274:H709-H718, 1998.
Cooke et al., J. Trauma, 60:363-370, 2006.
Cooke et al., Aviat. Space Environ. Med., 77:1107-1112, 2006.
Frankenhaeuser and Johansson, J. Human Stress, 2:15-23, 1976.
Godek et al., Br. J. Sports Med., 39:205-211, 2005.
Guézennec et al., Eur. J. Appl. Physiol., 68:525-530, 1994.
Holmes et al., J. Appl. Physiol., 92:2578-2584, 2002.
Hon and Lee, Am. J. Obstet. Gynecol., 87:814-826, 1963.
Jouanin et al., Mil. Med., 169:583-587, 2004.
Korach et al., Crit. Care Med., 29:1380-1385, 2001.
Krueger, G. P., Handbook of Military Psychology, Gal R and Mangelsdorff A D, eds., Chichester, England; John Wiley and Sons, Ltd. 244-277.
McLellan et al., Aviat Space Environ Med., 76: 647-654, 2005.
McNair et al., In: Profile of mood states manual, San Diego, Educational and Testing Service, 1971.
Nadel et al., J. Appl. Physiol., 49:715-721, 1980.
Nakano et al., Psychiatry Clin. Neurosci., 54:280-282, 2000.
Nindl et al., Med. Sci. Sports Sci., 39:1380-1387, 2007.
Norris et al., J. Surg. Res., 129:122-128, 2005.
Nose et al., Jpn. J. Physiol., 33:1019-1029, 1983.
Pichot et al., Med. Sci. Sports Exerc., 34:1660-1666, 2002.
Pinheiro and Bates, Statistics and Computing, 6:289-296, 1996.
Radespiel-Tröger et al., Clin. Auto. Res., 13: 99-102, 2003.
Riordan et al., J. Surg. Res., 156:283-289, 2009.
Ruby et al., Med. Sci. Sports Exerc., 35:1760-1765, 2003.
Task Force of the European Society of Cardiology and the North American Society of Pacing and Electrophysiology, Circulation, 93:1043-1065, 1996.
Tharion et al., Aviat Space Environ Med, 74: 309-314, 2003.
Tomasi et al., Impairment of attentional networks after 1 night of sleep deprivation. J Cereb Cortex 19: 233-240, 2009.
Winchell and Hoyt, J. Trauma, 43:927-933, 1997.

1. A method for quantifying fatigue of a subject, the method comprising:
measuring an electrocardiogram (ECG) signal from the subject;
calculating, with a processing device, a Heart Rate Variability (HRV) metric in response to the ECG signal; and
calculating, with a processing device, a fatigue level in response to the HRV metric.

2. The method of claim 1, further comprising transmitting the ECG signal to the processing device after measuring the ECG signal from the subject.

3. The method of claim 1, further comprising triggering an alarm in response to the fatigue level.

4. The method of claim 3, further comprising subjecting the subject to a stressor and assessing change in the HRV metric versus decline in cognitive performance.

5. The method of claim 1, wherein calculating the HRV metric comprises determining the average R-R interval over a period of time.

6. The method of claim 5, wherein the period of time is 30 seconds to 15 minutes.

7. The method of claim 1, wherein calculating the HRV metric comprises determining the R-R interval standard deviation over a period of time.

8. The method of claim 7, wherein the period of time is 30 seconds to 15 minutes.

9. The method of claim 1, wherein calculating the HRV metric comprises calculating the power spectral density of the ECG signal.

10. The method of claim 9, wherein calculating the power spectral density comprises:
filtering the ECG signal with a low-pass impulse response filter to form a filtered ECG signal; and
performing a Fourier transform on the filtered ECG signal to form a processed ECG signal.

11. The method of claim 10, the low-pass impulse response filter having a cut-off frequency of 0.5 Hz.

12. The method of claim 10, the Fourier transform comprising a Hanning window.

13. The method of claim 9, wherein calculating the HRV metric comprises calculating the power spectral density of the ECG signal across a frequency range from 0.04 Hz to 0.15 Hz.

14. The method of claim 9, wherein calculating the HRV metric comprises calculating the power spectral density of the ECG signal across a frequency range from 0.15 Hz to 0.4 Hz.

15. The method of claim 1, wherein measuring the ECG signal comprises an analog to digital conversion.

16. An apparatus for quantifying fatigue of a subject, the apparatus comprising:
two or more electrocardiogram (ECG) measuring pads configured to measure an ECG signal from a subject; and
a processing device, the processing device configured to:
calculate a Heart Rate Variability (HRV) metric in response to the ECG signal; and
calculate a fatigue level in response to the HRV metric;
wherein the ECG measuring pad and the processing device are comprised in a strap or pad, the ECG measuring pad configured to be positioned in contact with a surface of the subject.

17. The apparatus of claim 16, further comprising a transmitting device configured to send the ECG signal to the processing device.

18. The apparatus of claim 16, further comprising an alarm configured to trigger in response to the fatigue level.

19. (canceled)

20. The apparatus of claim 16, comprising two ECG measuring pads.

21. The apparatus of claim 16, wherein the two or more ECG measuring pads are comprised in a chest strap.

22-31. (canceled)

32. The apparatus of claim 16, further comprising an analog-to-digital converter.

33. A system for quantifying fatigue of a subject, the system comprising:
two or more electrocardiogram (ECG) measuring pads configured to measure an ECG signal from a subject; and
a processing device, the processing device configured to:
calculate a Heart Rate Variability (HRV) metric in response to the ECG signal; and
calculate a fatigue level in response to the HRV metric.

34. The system of claim 33, further comprising a transmitting device configured to send the ECG signal the processing device.

35. The system of claim 34, wherein the two or more ECG measuring pads and the transmitting device are comprised in a first strap or pad, the ECG measuring pad configured to be positioned in contact with a surface of the subject.

36. The system of claim 35, wherein the processing device is comprised in a second strap or pad.

37. The system of claim 33, wherein the processing device is comprised in a personal computing device.

38. The system of claim 33, further comprising an alarm configured to trigger in response to the fatigue level.

39. (canceled)

40. The system of claim 33, the two or more ECG measuring pads are comprised in a chest strap.

41-50. (canceled)

51. The system of claim 33, further comprising an analog to digital converter.
